(19) United States
(12) Patent Application Publication
(10) Pub. No.: US 2025/0302731 A1
(43) Pub. Date: Oct. 2, 2025

(54) Antioxidant Compositions

(71) Applicant: THE BOOTS COMPANY PLC, Nottingham (GB)

(72) Inventors: Mark Johnson, Nottingham (GB); Michael David Bell, Alfreton, Derbyshire (GB); Malcolm Kinninmonth, Derby (GB); Yegor Doush, West Bridgford, Nottingham (GB); Beverley Jane Elms, Nottingham, Leicestershire (GB)

(21) Appl. No.: 18/839,080

(22) PCT Filed: Feb. 16, 2023
(86) PCT No.: PCT/EP2023/025069

§ 371 Date: Aug. 16, 2024

(30) Foreign Application Priority Data
Feb. 17, 2022 (EP) 22020060.4

(51) Int. Cl. A61K 8/9789; A61K 8/9767; A61Q 19/00

(52) U.S. Cl. CPC A61K 8/9789; A61K 8/9767; A61Q 19/00; A61K 2800/10

(57) ABSTRACT

A cosmetic composition comprising the antioxidant agents: Myrtus communis leaf extract, Quercus petraea fruit extract and Pinus pinaster bark extract, and cosmetic methods of treatment and uses thereof.

### CROSS-REFERENCE TO RELATED APPLICATIONS

This application is a U.S. national stage of International Application No. PCT/EP2023/025069, filed Feb. 16, 2023, which claims priority to European Patent Application No. 22020060.4 filed Feb. 17, 2022.

### FIELD OF THE INVENTION

The present invention relates to cosmetic compositions providing improved skin protection and methods of cosmetic treatment using said compositions.

### BACKGROUND OF THE INVENTION

The skin is the first line of defence, serving as a barrier between us and the environment. The skin is a complex organ consisting of three layers: the epidermis, dermis and hypodermis.

The epidermis is the outermost layer, which itself is made up of several layers. The outermost portion of the epidermis, known as the stratum corneum, is relatively waterproof and, when undamaged, prevents most bacteria, viruses, and other foreign substances from entering the body. It also prevents the loss of moisture, heat and other important constituents of the body.

Most of the cells (90-95%) in the epidermis are keratinocytes. They originate from proliferating keratinocyte stem cells in the deepest layer of the epidermis called the basal layer. Resulting keratinocytes further divide and differentiate and slowly migrate up toward the surface of the epidermis as mature cells. Once the keratinocytes reach the stratum corneum at the skin surface they are dead and no longer multiplying and are gradually shed and replaced by newer cells pushed up from below.

The skin is subject to constant attack by a variety of both exogenous and endogenous insults. Exogenous insults include those arising from the environment such as ultraviolet radiation (UVA and UVB), infra-red and visible light, atmospheric pollution (including cigarette smoke) and/or harsh chemicals including surfactants in cosmetic formulations. Such environmental factors may either directly or indirectly result in skin damage by the generation of reactive species and free radicals, for example superoxide anions, hydrogen peroxide, hydroxyl ions, peroxyl ions, ozone, singlet oxygen, sulphur oxide, nitrogen oxide, carbon monoxide, alkoxyl ion, peroxynitrite and heavy metals. Reactive oxygen species (ROS), reactive carbonyl species (RCS) and reactive nitrogen species (RNS) need to be particularly considered. Endogenous insults can also result in skin damage, for example hormonal fluctuations (e.g. cortisol and adrenaline hormones), aging and other biochemical changes from within the skin.

As we age, our skin undergoes changes such as becoming thinner, more easily damaged and less elastic. In addition, lifetime exposure to UV-A ad UV-B radiation together with other environmental factors, that induce the formation of free radicals, such as pollution from traffic fumes, ozone, cigarette smoke etc., causes changes to the skin. These changes, including lines and wrinkling, actinic lentigines, dyspigmentation, rough skin, actinic telangiectasia and further loss of skin elastic function are due to direct UV-mediated damage to cells and indirectly mediated damage caused by the generation of free radicals in cells and tissues. This is generally termed photoaging and can account for up to 90% of the skin changes we associate with ageing.

The deleterious effects of UV radiation are generally believed to be due to the creation of free radicals. These highly reactive species may react with and damage DNA molecules in the skin (or elsewhere). Similar effects can also be attributed to radiation in the visible part of the spectrum.

With respect to atmospheric pollution (including cigarette smoke), polycyclic aromatic hydrocarbons (PAHs) are key pollutants that cause skin damage through a number of different mechanisms including increased melanocyte activation, increased sebum oxidation and mitochondrial damage of keratinocytes and fibroblasts. PAHs can also increase ROS discussed above in the skin.

The process of keratinocyte cell proliferation, differentiation and maturation is vulnerable to the many exogenous and endogenous insults that the skin faces on a daily basis. These insults are known to increase the inflammatory response in the epidermis. One consequence of this inflammation is the increased proliferation of keratinocytes followed by poor maturation and differentiation thereof, resulting in a lower quality stratum corneum and thus skin barrier disruption and/or damage. Once the skin barrier has been disrupted or damaged this further enhances the cascade of inflammation and keratinocyte over proliferation, creating a cycle of unhealthy skin traits. The skin barrier is weakened to the attack of pathogens and toxins, increasing the likelihood of skin redness and irritation, pimples and spots and/or causing the skin to appear dull, dry and scaly.

It is known to use antioxidant compounds as free radical quenchers, thereby mitigating the effects of free radical formation.

The Applicants have identified a consumer need to provide further cosmetic compositions which maintain or improve skin/hair health and/or appearance. The Applicants have found that the compositions of the present invention provide good and effective benefits to skin and hair against free-radical induced damage.

### SUMMARY OF THE INVENTION

Accordingly, in a first aspect of the invention, there is provided a cosmetic composition comprising the antioxidant agents: Myrtus communis leaf extract (such as that sold under the trade name of DEXOSINE BIO® from Silab), Quercus petraea fruit extract (such as that sold under the trade name of Phytessence French Oak™ from Croda), and Pinus pinaster bark extract (such as that sold under the trade name of Pycnogenol).

Surprisingly, the combination of specific antioxidant agents provide better antioxidant activity than the individual agents alone.

In another aspect of the invention there is provided a method of cosmetic treatment of a skin/hair condition comprising the step of applying the cosmetic composition according to the invention onto the skin/hair of a subject in need thereof.

In another aspect of the invention, there is provided a use of the cosmetic composition according to the invention as a topical application on the skin/hair.

It is appreciated that the cosmetic compositions of the present invention can be effective in cosmetically treating skin damage as a result of pollution insult or cosmetically preventing the detrimental effects of pollution insult to the skin. Thus, a further aspect of the present invention provides a method of cosmetically treating skin damage as a result of pollution insult, or of cosmetically preventing the detrimental effects of pollution to the skin, said method comprising applying an effective amount of the cosmetic composition defined above to the skin.

### DETAILED DESCRIPTION OF THE INVENTION

The invention makes use of antioxidant agents. The term “antioxidant agent” is intended to mean an agent that inhibits oxidation and prevents the formation of free radicals as would be understood by a person skilled in the art.

The invention makes use of an antioxidant agent Myrtus communis leaf extract (such as that sold under the trade name of DEXOSINE BIO® from Silab).

One of the antioxidant agents is Quercus petraea fruit extract (such as that sold under the trade name of Phytessence French Oak™ from Croda).

One of the antioxidant agents is Pinus pinaster bark extract (such as that sold under the trade name of Pycnogenol).

The antioxidant agents Myrtus communis leaf extract (such as that sold under the trade name of DEXOSINE BIO® from Silab), Quercus petraea fruit extract (such as that sold under the trade name of Phytessence French Oak™ from Croda), and Pinus pinaster bark extract (such as that sold under the trade name of Pycnogenol) may be present in an amount of about 0.0001% to about 10% by weight the composition, about 0.0003% to about 1% by weight of the composition, about 0.0005% to about 0.5% by weight of the composition, about 0.0006% to about 0.1% by weight of the composition, about 0.0007 to about 0.01% by weight of the composition, about 0.0008% to about 0.005% by weight of the composition, about 0.0009% to about 0.0025% by weight of the composition.

The antioxidant agents may be present in the composition in the same/equal amounts or approximately the same/equal amounts.

One antioxidant agent may present in an amount of at least the same amount as the other two antioxidant agents. In one embodiment, the first antioxidant agent is present in an amount of about 4 times or less the amount of the other antioxidant agents, for example about 3.5 times or less, about 3 times or less, or about 2.5 times or less.

In one embodiment, the antioxidant composition may comprise an additional antioxidant agent or agents. The additional antioxidant agent(s) may be a plant, algal or fungal extract, or derivative thereof, comprising one or more species which provide an antioxidant benefit, such as flavonoid species; phenolic acid species; stilbene species; lignin species, or combinations thereof. Alternatively, the additional antioxidant agent(s) may be synthetic or nature identical.

In one embodiment, the additional antioxidant agent(s) comprises one or more flavonoid species. Flavonoid species include flavones, flavonols, flavanones, flavanols, anthocyanidins, anthocyanins, proanthocyanidins, flavans, isoflavones and isoflavonoids. Some specific examples of flavonoid species are catechins (catechin, epicatechin, gallocatechin, epigallocatechin, epicatechin gallate, epigallocatechin gallate), quercetin, rutin, hesperidin and genistein.

In one embodiment, the additional antioxidant agent(s) comprises one or more carotenoid species. Carotenoid species include xanthophylls and carotenes. Specific examples of carotenoids are β-carotene, alpha-carotene, gamma carotene, beta-cryptoxanthin, lycopene, lutein, and zeaxanthin.

In one embodiment, the additional antioxidant agent(s) comprises one or more enzyme species. Antioxidant enzymes include glutathione peroxidases, catalases and superoxide dismutase (SOD).

In one embodiment, the additional antioxidant agent(s) comprises one or more glutathione species. Glutathione species include reduced glutathione (CSH, or L-glutathione), and oxidized glutathione (GSSG), the inactive state.

In one embodiment, the additional antioxidant agent(s) comprises one or more lipid associated chemical species. Lipid associated chemical species include Ubiquinol-10, N-acetyl cysteine, lipoic acid.

In one embodiment, the additional antioxidant agent(s) comprises one or more saponine or steroid species. Saponin species may be plant-derived, or isolated from marine organisms such as sea cucumber. Examples of saponin sources are Sapindaceae, including its defining genus Sapindus (soapberry or soapnut), Aesculus hippocastanum (horse chesnut), Aceraceae (maples), Hippocastanaceae, Gynostemma pentaphyllum (Cucurbitaceae), ginseng or red ginseng (Panax, Araliaceae) Manilkara zapota fruit (also known as sapodillas) and Nerium oleander (Apocynaceae). Saponins may be derived from the leaves, stems, roots, bulbs, blossom and fruits of these plants.

In one embodiment, the additional antioxidant agent(s) comprises one or more vitamins, such as vitamin C or vitamin E.

Plants provide a rich and cheap source of antioxidant agents and are therefore an efficient source of said agents. Naturally occurring antioxidant agents may therefore be used.

However, the same or similar actives can be also prepared synthetically. The term “antioxidant agent” is therefore intended to cover antioxidant agents including synthetic polyphenols, such as synthetic analogues of naturally occurring antioxidant agents. Thus chemically synthesized or purified polyphenols and mixtures thereof may be used in place of plant extracts. Polyphenols may be synthesized or extracted from natural sources by any suitable method known to those skilled in the art, particularly using food-grade solvents. Liquid and solid (e.g. granulate or powder form) extracts are suitable.

Extracts (e.g. aqueous or alcoholic) can be obtained from plant parts including but not limited to leaves, raw or cooked whole fruit, berries and vegetables, nuts, the skins of fruit, fruit flesh, fruit rind, peel, pips, cones (e.g. hops), seeds or stones, bark, buds, flowers or parts thereof, including petals and pollen, roots, rhizomes and tubers, and stems. The plant extract may be selected from the group consisting of essential oils, extracts from leaves, extracts from stems, extracts from petals, extracts from seeds, extracts from roots, extracts from pollen, and combinations thereof. In one embodiment, extracts (e.g. lyophilised extracts) from leaves are used. In one embodiment, extracts may be prepared by microwave extraction (e.g., Quercus petraea fruit extract).

The term “antioxidant agent” is intended to mean a plant extract or derivative thereof comprising flavonoid species, including flavones, flavonols, flavanones, flavanols, anthrocyanidins and isoflavonoids; phenoic acid species; stilbenes; lignans or combinations thereof, which provides an antioxidant benefit. Plants provide a rich and cheap source of antioxidant agents and are therefore an efficient source of said agents. Similar actives can be also prepared synthetically and as such are analogues. The term “antioxidant agent” is also intended to cover said analogues. The plant extract may be selected from the group consisting of essential oils, extracts from leaves, extracts from stems, extracts from petals, extracts from seeds, extracts from roots, extracts from pollen, and combinations thereof.

The additional antioxidant agent(s) may be selected from the group consisting of dimethylmethoxy chromanol (such as that sold under the trade name of LIPOCHROMAN® Molecule from Lipotec), green tea extract (e.g. Camellia sinensis leaf extract), Camellia japonica extract (e.g. a Camellia japonica flower extract such as that sold under the trade name of RedSnow® from Clariant), emblica extract (e.g. Phyllanthus emblica fruit extract (also known as Emblica officinalis (Gooseberry) fruit extract)), Argania spinosa leaf extract (such as that sold under the trade name of Arganyl™ from BASF), Resveratrol (such as that sold under the trade name of REGU®-FADE), and combinations thereof.

The additional antioxidant agent(s) (when present) may (each) be present in an amount of about 0.0001% to about 10% by weight the composition, about 0.0003% to about 1% by weight of the composition, about 0.0005% to about 0.5% by weight of the composition, about 0.0006% to about 0.1% by weight of the composition, about 0.0007 to about 0.01% by weight of the composition, about 0.0008% to about 0.005% by weight of the composition, about 0.0009% to about 0.0025% by weight of the composition.

The antioxidant agents may be present in an amount of about 0.001% to about 10% by weight the composition, about 0.003% to about 1% by weight of the composition, about 0.005% to about 0.5% by weight of the composition, about 0.006% to about 0.2% by weight of the composition, about 0.007% to about 0.1% by weight of the composition, about 0.0008% to about 0.005% by weight of the composition, about 0.005% to about 0.01% by weight of the composition.

The antioxidant agents may be present in an amount of about 0.01% to about 5% by weight the composition, about 0.05% to about 3% by weight of the composition, about 0.1% to about 2% by weight of the composition, about 0.2% to about 1.8% by weight of the composition, about 0.4% to about 1.6% by weight of the composition, about 0.6% to about 1.4% by weight of the composition, about 0.8% to about 1.2% by weight of the composition.

The antioxidant agents may be present in an amount of about 0.1% to about 10% by weight the composition, about 0.5% to about 7% by weight of the composition, about 1% to about 5% by weight of the composition, about 1.2% to about 4.8% by weight of the composition, about 1.5% to about 4.5% by weight of the composition, about 1.8% to about 4.2% by weight of the composition, about 2% to about 4% by weight of the composition.

The cosmetic composition of the invention may comprise a cosmetically acceptable carrier. The cosmetically acceptable carrier may be water-based, oil- or wax-based, or emulsion-based.

In embodiments where the carrier is emulsion-based, the composition may be in the form of a water-in-oil, an oil-in-water, a water-in-oil-in-water or a oil-in-water-in-oil emulsion.

In embodiments where the carrier is water-based, water may be present at a level of about 40% or more, about 45% or more, about 50% or more, about 55% or more, or about 60% or more by weight of the composition.

In embodiments where the carrier is oil- or wax-based, the oil and/or wax may be present at a level of about 15% or more, about 20% or more, about 30% or more, about 25% or more, about 35% or more, or about 40% or more by weight of the composition.

For example, in one embodiment the carrier may be water based and may comprise de-ionized water, purified water, natural spring water, rose water or the like. Mixtures of more than one of these may also be used. In one embodiment de-ionized or purified water is used.

The water based carrier may be 100% water or it may comprise components other than water. These may be components known for use in cosmetic formulations. They may include, but are not limited to, agents such as water-soluble moisturising agents, conditioning agents, anti-microbials, humectants (e.g. glycerin) and/or other water-soluble skin care actives.

In another embodiment, the carrier may be oil or wax based. The oil may be natural oil or synthetic oil, but preferably is natural oil such as a vegetable oil or a nut oil. The oil may be liquid or solid. The wax is preferably a natural wax.

Clearly the oil or wax that is chosen must be able to act as a carrier. Preferably it is a material that can easily be blended at room temperature; thus it may be a liquid at room temperature or a solid that is stirrable at room temperature.

Combinations of one or more oils and/or one or more waxes may be used.

Liquid oils that can be mentioned include avocado oil, Camellia oil, turtle bean oil, macadamia nut oil, corn oil, mink oil, olive oil, Canoga oil, egg yolk oil, sesame seed oil, Persic oil, wheat germ oil, Camellia sasanqua oil, castor oil, linseed oil, safflower oil, sunflower oil, grapeseed oil, apricot oil, shea oil, sweet almond oil, cotton oil, evening primrose oil, palm oil, perilla oil, hazelnut oil, soybean oil, peanut oil, tea seed oil, kaya oil, rice bran oil, rapeseed oil, alfalfa oil, Chinese tung tree wood oil, Japanese tung tree wood oil, jojoba oil, germ oil, poppyseed oil, pumpkin oil, blackcurrant oil, millet oil, barley oil, quinoa oil, rye oil, candlenut oil, passionflower oil, musk rose oil, triglycerine, glyceryl trioctanoate, and glyceryl triisopalmitate.

Solid oils/fats that can be mentioned include cocoa butter, coconut butter, horse fat, hardened coconut oil, palm oil, beef tallow, mutton tallow, hardened beef tallow, palm kernel oil, lard, Japan wax kernel oil, hardened oil, Japan wax, shea butter, and hardened castor oil;

Waxes that can be mentioned include beeswax, candelilla wax, carnauba wax, lanolin, lanolin acetate, liquid lanolin, sugar cane wax, fatty acid isopropyl lanolin, hexyl laurate, reduced lanolin, jojoba wax, hard lanolin, polyoxyethylene (hereinafter referred to as POE), lanolin alcohol ether, POE lanolin alcohol acetate, lanolin fatty acid polyethylene glycol, and POE hydrogenated lanolin alcohol ether. In one embodiment the carrier is not lanolin based.

Ester oils that can be mentioned include C12-C15 alcohols benzoate, tridecyl salicylate, dibutyl adipate, isopropyl myristate, cetyl octoate, octyldodecyl myristate, isopropyl palmitate, butyl stearate, hexyl laurate, myristyl myristate, decyloleate, hexyldecyl dimethyl octoate, cetyl lactate, myristyl lactate, lanolin acetate, isocetyl stearate, isocetyl iso-stearate, 12-hydroxy cholesteryl stearate, di-2-ethylhexylic acid ethyleneglycol, dipentaerythritol fatty acid ester, N-alkylglycol monoisostearate, neopentylglycol dicaprate, diisostearyl malate, glyceryl di-2-heptyl undecanate, tri-methylol propane tri-2-ethylhexyl acid, tri-methylol propane triisostearate, pentaerythritol tetra-2-ethylhexyl acid, glyceryl tri-2-ethyl-hexanoate, tri-methylol propane triisostearate, cetyl-2-ethylexanoate, 2-ethylhexyl-palmitate, glycerine trimyristate, glyceride tri-2-heptyl undecatoic acid, methyl ester of castor oil fatty acid, oleate oil, acetoglyceride, palmitate-2-heptyl undecyl, diisopropyl adipate, N-lauroyl-L-glutamic acid-2-octyldodecil ester, di-2-heptylundecyl adipate, di-2-ethylhexyl sebacate, myristate-2-hexyldecyl, palmitate-2-hexyldecyl, adipate-2-hexyldecyl, diisopropyl sebacate, and succinate-2-ethylhexyl.

Higher fatty acids that can be mentioned include lauric acid, myristic acid, palmitic acid, stearic acid, behenic acid, oleic acid, 12-hydroxy-stearic acid, undecylenic acid, lanolin fatty acid, isostearic acid, linoleic acid, linolenic acid, and eicosapentaenoic acid.

Higher alcohols of straight/branched chain that can be mentioned include lauryl alcohol, cetyl alcohol, stearyl alcohol, behenyl alcohol, myristyl alcohol, oleyl alcohol, cetostearyl alcohol, monostearyl glycerine ether (batyl alcohol), 2-decyltetradecinol, lanolin alcohol, cholesterol, phytosterol, hexyldodecanol, isostearyl alcohol, octyldodecanol.

The cosmetic composition of the invention may be provided in any form suitable for topical application to the skin/hair. The cosmetic composition of the invention may be delivered and/or applied to the skin via any of the conventional formulations known to those skilled in the art. Typical formulation types of the present invention are creams, lotions, milks, gels, serum, foams, and ointments. In one embodiment, the cosmetic composition of the present invention is in the form of a cream or lotion.

The cosmetic composition of the invention will generally further comprise other ingredients or excipients which will be well known to those skilled in the art.

For example, the cosmetic composition of the invention may further comprise one or more humectants, including but not limited to glycerin, propylene glycol, propanediol, butylene glycol, pentylene glycol, hexylene glycol, hexanediol, dipropylene glycol, polyethylene glycol, sorbitol, sodium hyaluronate, urea, xylitol, lactitol, fructose, glucose, mannose, xylose, honey, pyrrolidone, and carboxylic acid and salts thereof. When present, the one or more humectants may be present in the cosmetic composition in an amount of about 0.01% to about 20% by weight of the composition, about 0.1% to about 10%, or about 0.5% to about 7% by weight of the composition.

The cosmetic composition of the invention may further comprise one or more emollients, including but not limited to PPG-15 stearyl ether, ethylhexyl stearate, cetyl dimethicone, octyldodecanol, PPG-20 methyl glucose ether, isopropyl myristate, isopropyl paltimate, isopropyl laurate, isodecyl laurate, isodecyl neopentanoate, isohexadecane, pentaerythrityl tetraisostearate, caprylic/capric triglyceride, canola oil, sunflower oil (Helianthus annus), olive oil (Olea europea), cottonseed oil (Gossypium herbaceum), jojoba oil (Simmondsia chinensis), shea butter (Butyrospermum parkii), cocoa butter (Theobroma cacao), cupuacu butter (Theobroma grandiflorum), avocado oil (Persea gratissima), liquid paraffin, dimethicone, phenyl trimethicone, cyclopentasiloxane, dimethiconol and petrolatum. When present, the one or more emollients may be present in the cosmetic composition in an amount of about 0.01% to about 20% by weight of the composition, about 0.1% to about 10%, or about 0.5% to about 7% by weight of the composition.

The cosmetic composition may further comprise one or more emulsifiers, including but not limited to steareth-2, steareth-21, steareth-10, ceteareth-5, ceteareth-20, cetearyl glucoside, oleth-10, glyceryl stearate, polyglycerol-3 oleate, polyglyceryl-3 methylglucose distearate, sodium stearate, PEG-12 oleate, PEG-2 stearate, PEG-12 stearate, PEG-100 stearate, cetyl alcohol, cetearyl alcohol, potassium cetyl phosphate, cetearyl olivate, sorbitan olivate, PEG-80 sorbitan, sorbitan oleate, and/or sorbitan palmitate. In one embodiment, the cosmetic composition of the invention does not comprise sulphates as emulsifiers. In embodiments where one or more emulsifiers are present in the cosmetic composition, the one or more emulsifiers may be present in an amount of about 0.1% to about 10% by weight of the composition, about 0.25% to about 7.5% by weight of the composition, or about 0.5% to about 5% by weight of the composition. In one embodiment where one or more emulsifiers are present in the cosmetic composition, the one or more emulsifiers are present in an amount of about 0.5% to about 5% by weight of the composition.

The cosmetic composition of the invention may further comprise one or more surfactants, including but not limited to, anionic surfactants (e.g. sodium lauryl sulphate, sodium laureth sulphate, ammonium laureth sulphate, disodium laureth sulfosuccinate and sodium C12-15 pareth-12 carboxylate), amphoteric/zwitterionic surfactants (e.g. cocamidopropyl betaine, sodium cocoamphoacetate and cocamidopropyl hydroxysultaine), non-ionic surfactants (e.g. cocamide DEA, cocamide MEA, decyl glucoside, lauryl glucoside), and cationic surfactants (e.g. cetrimonium chloride, behentrimonium chloride and benzalkonium chloride). In one embodiment, the cosmetic composition of the invention does not comprise sulphates as surfactants. In embodiments where one or more surfactants are present in the cosmetic composition, the one or more surfactants may be present in an amount of about 0.1% to about 10% by weight of the composition, about 0.25% to about 7.5% by weight of the composition, or about 0.5% to about 5% by weight of the composition. In one embodiment where one or more surfactants are present in the cosmetic composition, the one or more surfactants are present in an amount of about 0.5% to about 5% by weight of the composition.

The cosmetic composition of the invention may further comprise one or more preservatives, including but not limited to, 2-bromo-2nitropropane-1,3-diol (bronopol, commercially available under the trade name Myacide®), benzyl alcohol, benzoic acid, sodium benzoate, diazolidinyl urea, imidazolidinyl urea, methyl paraben, phenoxyethanol, ethyl paraben, propyl paraben, sodium methyl paraben, sodium dehydroacetate, dehydroacetic acid, polyhexamethylenebiguanide hydrochloride, isothiazolone, chlorhexidine digluconate, chlorphensin and/or sodium propyl paraben. In one embodiment, the cosmetic composition of the invention does not comprise parabens. In embodiments where one or more preservatives are present in the cosmetic composition, the one or more preservatives may be present in an amount of about 0.001% to about 10% by weight of the composition, about 0.01% to about 8% by weight of the composition, or about 0.1% to about 5% by weight of the composition. In one embodiment where one or more preservatives are present in the cosmetic composition, the one or more preservatives are present in an amount of about 0.05% to about 8% by weight of the composition.

The cosmetic composition of the invention may further comprise one or more chelating agents or sequestering agents, including but not limited to, ethylenediamine tetraacetic acid (EDTA) and salts thereof (e.g. dipotassium EDTA, disodium EDTA or tetrasodium EDTA), sodium phytate, trisodium ethylene diamine disuccinate and/or tetrasodium glutamate diacetate. In embodiments where one or more chelating agents are present in the cosmetic composition, the one or more chelating agents may be present in an amount of about 0.001% to about 10% by weight of the composition, about 0.01% to about 8% by weight of the composition, or about 0.1% to about 5% by weight of the composition. In one embodiment where one or more chelating agents are present in the cosmetic composition, the one or more chelating agents are present in an amount of about 0.05% to about 8% by weight of the composition.

The cosmetic composition of the invention may further comprise one or more vitamins. For example, the cosmetic composition may further comprise vitamin B, vitamin Bi to vitamin B12, vitamin C, vitamin D, vitamin E, vitamin K, vitamin H, derivatives thereof, provitamins thereof (e.g. pro-vitamin B5 (panthenol)), or combinations thereof. In embodiments where one or more vitamins are present in the cosmetic composition, the one or more vitamins may be present in a amount of about 0.0001% to about 50% by weight of the composition, about 0.001% to about 10% by weight of the composition, about 0.01% to about 8% by weight of the composition, or about 0.1% to about 5% by weight of the composition. In one embodiment where one or more vitamins are present in the cosmetic composition, the one or more vitamins are present in an amount of about 0.1% to about 5% by weight of the composition. In one embodiment where one or more vitamins are present, the vitamin is vitamin C, vitamin E and/or a derivative thereof.

The cosmetic composition of the invention may further comprise one or more antioxidants. These may be different to the antioxidant agents already present in the composition. For example, the cosmetic composition may further comprise one or more of extracts of: mulberry (e.g. Morus alba), ginseng (e.g. Panax ginseng), raspberry, oregano (e.g. Origanum vulgare), white tea (e.g. Camellia sinensis), red tea, Mohani tea, black tea, Oolong tea, yellow tea, jasmine tea, Pu Erh tea, blueberry (e.g. Vaccinium cyanococcus), rosemary (e.g. Rosmarinus officialis), grape, including grape seed (e.g. Vitis vinifera), fennel (e.g. Foeniculi fructus), Caragana sinica, majaoram (e.g. Origanum majorana), crocus (e.g. Crocus sativus), apple (e.g. Malus domestica), coffee, green coffee, cherry (e.g. Prunus avium), snow algae (e.g. Chlamydomonas nivalis), moringa (e.g. Moringa oleilera), ginger, magnolia (e.g. Magnolioideae virginiana), French saffron, edelweiss (e.g. Leontopodium alpinium), white lotus (e.g. Nymphaea alba), turmeric root, marshmallow (e.g. Althaea officianlis), burdock (e.g. Arctium lappa), bilberry (e.g. Vaccinium myrtillus), cranberry (e.g. Vaccinium oxycoccus), pomegranate (e.g. Punica granatum), sage (e.g. Salvia officianlis), thyme (e.g. Thymus vulgaris), sunflower (e.g. Helianthus annus), wild carrot (e.g. Daucus carota), hop (e.g. Humulus lupulus), witch hazel (e.g. Hamamelis), oak (e.g. Quercus), Camellia (e.g. Theacea), red clover (e.g. Tritolium pratense), flax (e.g. Linium usitatissiumum), lemon (e.g. Citrus limon), birch (e.g. Betula), cornflower (e.g. Centaurea cyanus), geranium, polygonum, soy (e.g. Glycine max), or Sophora (e.g. Sophora flavescens). In one embodiment where one or more such additional antioxidants are present in the cosmetic composition, the one or more additional antioxidants are present in an amount of from about 0.1% to about 5% by weight of the composition.

The cosmetic composition of the invention may further comprise one or more sunscreen agents, including but not limited to inorganic sunscreen agents (e.g. microfine titanium dioxide, microfine zinc oxide, iron oxides, tales and/or boron nitride) and organic sunscreen agents (e.g. p-aminobenzoic acids, esters and derivatives thereof (e.g. 2-ethylhexyl p-dimethyl-aminobenzoate), methoxycinnamate esters (e.g., 2-ethylhexyl p-methoxycinnamate, 2-ethoxyethyl p-methoxycinnamate or α,β-di-(p-methoxycinnamoyl)-α′-(2ethylhexanoyl)-glycerin), benzophenones (e.g. oxybenzone), dibenzoylmethanes (e.g. 4-(tert-butyl)-4′-methoxydibenzoylmethane), 2-phenylbenzimidazole-5 sulfonic acid and salts thereof, alkyl-β,β-diphenylacrylates (e.g. alkyl α-cyano-D,D-diphenylacrylates such as octocrylene) triazines (such as 2,4,6-trianilino-(p-carbo-2-ethyl-hexyl-1-oxy)-1,3,5 triazine), and/or camphor derivatives (such as methylbenzylidene camphor). In embodiments where one or more sunscreen agents are present in the cosmetic composition, the one or more sunscreen agents may be present in an amount of about 0.01 to about 10% by weight of the composition.

The cosmetic composition of the invention may further comprise one or more pH adjusting agents, including but not limited to potassium hydroxide, sodium hydroxide, aminomethyl propanol, sodium citrate and/or triethanolamine. The cosmetic composition of the invention may have a pH from about 3 to about 10, e.g. from about 4 to about 8, or from about 5 to about 7. In embodiments where one or more pH adjusting agents are present in the cosmetic composition, the one or more pH adjusting agents may be present in an amount of from about 0.01 to about 10% by weight of the composition.

The cosmetic composition of the invention may further comprise one or more thickeners or gelling agents. For example, when the cosmetic composition is in the form of a gel, the cosmetic composition may comprise one or more thickeners or gelling agents. Examples of thickeners/gelling agents that can be used in the present invention include, but are not limited to, acrylic acid polymers (e.g. available commercially under the trade name Carbopol or Ultrez (Lubrizol), modified celluloses (e.g. hydroxyethylcellulose available commercially under the trade name Natrosol from Hercules) hydroxypropylmethyl cellulose, amine oxides, block polymers of ethylene oxide and propylene oxide (e.g. those available from BASF Wyandotte under the trade name “Pluronic”), PVM, MA, decadiene crosspolymer (e.g. available under the trade name Stabilez 60), ethoxylated fatty alcohols, salt (e.g. magnesium chloride, sodium chloride), Aristoflex AVC, phthalic acid amide, xanthan gum, sodium polyacrylate, polyvinyl alcohols, fatty alcohols, and/or alkyl galactmanans (e.g. available under the trade name N-Hance from Hercules. In embodiments where one or more thickeners/gelling agents are present in the cosmetic composition, the one or more thickeners/gelling agents may be present in an amount of about 0.01 to about 10% by weight of the composition.

The cosmetic compositions of the invention may further comprise one or more perfumes and/or colourings. The cosmetic composition may further comprise one or more conditioning agents.

The composition may also include waxes such as cocoa butter suitably in an amount of from 1% to 99% by weight of the composition. The composition may also comprise suitable, cosmetically acceptable diluents, carriers and/or propellants such as dimethyl ether. The composition may also include pearlising agents such as stearic monoethanolamide and/or mica, suitably in an amount of from 0.01% to 10% by weight of the composition

The cosmetic treatment may for alleviating or preventing the appearance of a skin condition selected from the group consisting of skin ageing, skin elastosis, skin laxity (sagging), rhytids (wrinkles), skin inflammation, skin damage, skin burn, skin pain, muscle tightness and acne.

In the present application, the term “about” may encompass ±10%, such as ±5%, e.g. ±2% or ±1%.

The skilled person will understand that optional features of one embodiment or aspect of the invention may be applicable, where appropriate, to other embodiments or aspects of the invention.

There now follows by way of example only a description of the present invention with reference to the accompanying drawings, in which:

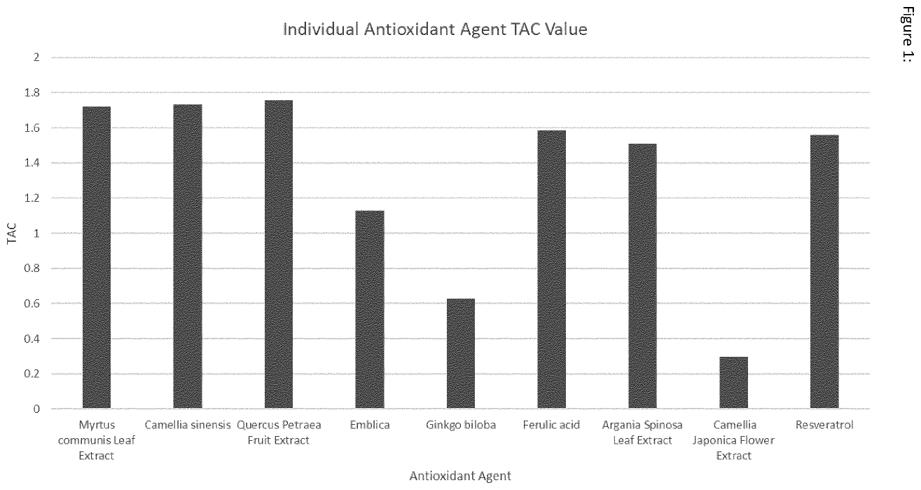

FIG. 1: The graph shows the Total Antioxidant Capacity (TAC) of each of the individual antioxidant agents.

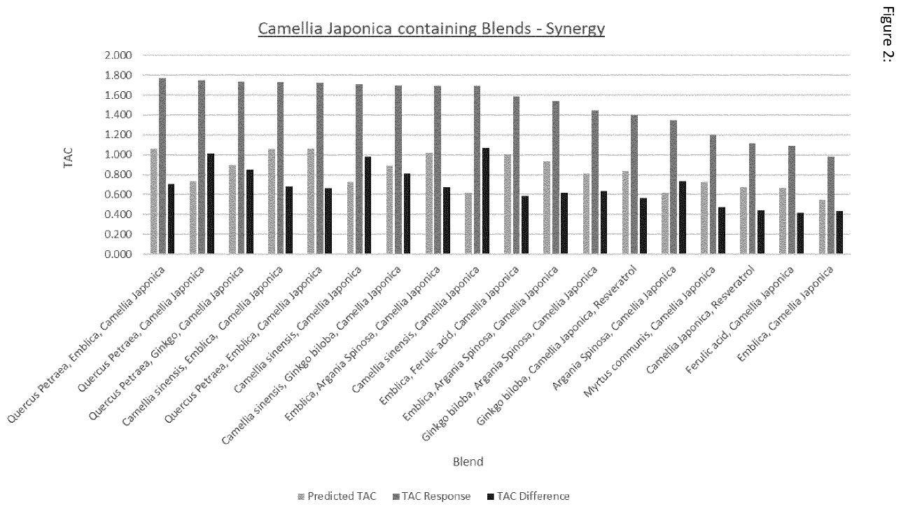

FIG. 2: The graph shows the actual TAC value for the antioxidant blends having the highest TAC values compared with the predicted TAC value based on the individual TAC values, and the difference between the actual and predicted values.

### EXAMPLES

### Total Antioxidant Capacity Assay

Total antioxidant capacity (TAC) was determined using a kit (Cell Biolabs, Inc) as follows; the test samples were diluted in water and a buffer to reach a final antioxidant agent to buffer ratio ranging from 1:1 to 1:1,000,000. 20 ul of test sample or control (ascorbic acid or green tea) were added to the wells of 96 well plate, followed by 180 μL of 1× Reaction Buffer (provided with the kit). The absorbance of the reaction mix in each well was measured at 490 nm to get an initial reading. 50 μL of the 1× Copper Ion Reagent (provided with the kit) were added to each well to initiate the reaction. The reaction was allowed to carry on for 5 minutes on an orbital shaker and 50 μL of 1× Stop Solution (provided with the kit) added to each well to terminate the reaction. The colour change was assessed using a plate reader at 490 nm.

The stronger the colour change at the end of the reaction, the higher the spectrophotometric value, and the higher the antioxidant capacity of the antioxidant agents.

### Example 1—Identifying Antioxidant Candidates

21 lead antioxidant compounds were tested to determine which had the highest Total Antioxidant Capacity. 21 antioxidant compounds were tested in triplicate at antioxidant: buffer ratios ranging from 1:1 to 1:1,000,000. The TAC for these candidates is shown in the table below.

| | | Concentration | | | | | | |
|---|---|---|---|---|---|---|---|---|
| | | 1 | 0.1 | 0.01 | 0.001 | 0.0001 | 0.00001 | 0.000001 |
| | | Antioxidant:Buffer | | | | | | |
| | | 1:1 | 1:10 | 1:100 | 1:1,000 | 1:10,000 | 1:100,000 | 1:1,000,000 |
| 1 | Dimethylmethylmethoxy | 1.839 | 1.807 | 1.824 | | | | |
| | chromanol | | | | | | | |
| 2 | Ascorbic Acid | 2.6 | 1.6 | 0.7 | | | | |
| | (Control) | | | | | | | |
| 3 | Scutellaria alpina | | | 0.2231 | 0.04798 | 0.02031 | | |
| | flower | | | | | | | |
| 4 | Sophora japonica | | | 0.7099 | 0.04900 | 0.02449 | | |
| | flower extract | | | | | | | |
| 5 | Vitis Vinifera (Grape) | | | 0.3222 | 0.04663 | 0.01232 | | |
| | Juice extract | | | | | | | |
| 6 | Myrus communis Leaf | | 0.8364 | 1.078 | 0.3042 | | | |
| | Extract | | | | | | | |
| 7 | Camellia sinensis leaf | 0.9353 | 1.876 | 2.317 | | | | |
| | extract | | | | | | | |
| 8 | Bacillus Ferment | | | | 0.2183 | 0.1068 | 0.03695 | |
| 9 | Dimethylmethoxy | 0.6546 | 0.9408 | 1.364 | | | | |
| | Chromal (Emulsion) | | | | | | | |
| 10 | Leontopodium | 1.109 | 0.1067 | 0.02850 | | | | |
| | alpinum callus | | | | | | | |
| | culture extract | | | | | | | |
| 11 | Quercus petraea fruit | | 2.044 | 2.092 | 0.2267 | | | |
| | extract | | | | | | | |
| 12 | Phyllantus emblica | 1.161 | 1.711 | 1.876 | | | | |
| | fruit extract. | | | | | | | |
| 13 | Ginkgo Biloba leaf | 0.6257 | 0.6314 | 1.379 | | | | |
| | extract | | | | | | | |
| 14 | Sparassis crispa | | 0.1670 | 0.08793 | 0.02799 | | | |
| | extract | | | | | | | |
| 15 | Argania spinosa leaf | | 1.467 | 0.6358 | 0.07094 | | | |
| | extract | | | | | | | |
| 16 | Litchi chinensis | | | | 0.2204 | 0.05789 | 0.01854 | |
| | pericarp extract | | | | | | | |
| 17 | Fullerenes | | | | | 0.05485 | 0.02915 | 0.01707 |
| 18 | Pinus pinaster back | 0.8071 | 1.196 | 1.040 | | | | |
| | extract | | | | | | | |
| 19 | Resveratrol | 1.883 | 1.709 | 1.689 | | | | |
| 20 | Ferulic acid | 1.911 | 1.808 | 1.991 | | | | |
| 21 | Camellia japonica | | 1.367 | 0.1943 | 0.03236 | | | |
| | flower extract | | | | | | | |

From these 21 compounds, 11 were selected as the most promising: Dimethylmethoxy chromanol, Myrtus communis leaf extract, Camellia sinensis leaf extract, Quercus petraea fruit extract, Phyllanthus emblica fruit extract, Ginkgo biloba leaf extract, Ferulic acid, Argania spinosa leaf extract, Pycnogenol, Camellia japonica flower extract and Resveratrol.

### Example 2—Formulating Antioxidant Compositions

The 11 lead antioxidant agents were combined into 330 different antioxidant compositions comprising 2 or 3 different antioxidant agents. compositions. Predicted TAC values for different antioxidant compositions were calculated using a custom experimental design created using the JMP DOE module with 11 antioxidants as mixture factors, Total Antioxidant Capacity (TAC) as the Y response, and modelling 3rd order interactions between the factors.

The experimental design defined 330 experiments which contained a minimum of 1 antioxidant and a maximum of 3 antioxidants for any given experiment.

Once the defined experiments had been run the results were modelled using a Stepwise Fit for Y model with the following regression controls. Stopping Rule: Minimum AICc, Direction: Forward, Rules: Combine.

The interactive mixture profiler module was used to predict the ratio combination that provided the maximum TAC for all possible mixtures of 3 antioxidants from the original list of 11 antioxidant factors.

The actual TAC of each of these compounds was calculated using the TAC assay described above and compared with the predicted values to determine the % difference in TAC. The TAC values for the top performing blended antioxidant compositions is shown in Table 1 below. FIG. 1 is a graph that shows the predicted TAC, actual TAC and the difference between the predicted and actual values.

A blend of Myrtus communis leaf extract, Quercus petraea fruit extract and Pinus pinaster bark extract had a particularly high TAC value.

Tested blends

| | Myrtus | Camellia | Quercus | Phyllanthys | Ginkgo | | Argania | Pinus | Camellia | | |
|---|---|---|---|---|---|---|---|---|---|---|---|
| % ratio of | communis | sinensis | Petraea | emblica | Biloba | | Spinosa | Pinaster | Japonica | | |
| actives in | Leaf | leaf | Fruit | fruit | Leaf | Ferullic | Leaf | Bark | Flower | | TAC |
| blend | Extract | extract | Extract | extract | Extract | acid | Extract | Extract | Extract | Resveratrol | value |
| Blend 1 | | | | | | | 33.33% | 33.33% | | 33.33% | 1.866 |
| Blend 2 | 33.33% | | | | 33.33% | 33.33% | | | | | 1.849 |
| Blend 3 | | | 33.40% | 33.40% | | | | | 33.21% | | 1.769 |
| Blend 4 | 33.33% | | 33.33% | | | | | 33.33% | | | 1.765 |
| Blend 5 | | 33.33% | 33.33% | | | 33.33% | | | | | 1.76 |
| Blend 6 | | 33.33% | 33.33% | | | | | | 33.33% | | 1.759 |

### Representative Formulations

Example 1 - Water in oil emulsion

| Material | % w/w | % w/w | % w/w |
|---|---|---|---|
| Dimethicone | 13.83 | 13.83 | 13.83 |
| Water | 37.01 | 34.91 | 36.31 |
| Glycerin | 5.00 | 5.00 | 5.00 |
| Dimethicone crosspolymer & | 32.31 | 32.31 | 32.31 |
| Dimethcone | | | |
| Butylene glycol | 2.60 | 2.60 | 2.60 |
| PEG/PPG-18/18 dimethicone & | 3.00 | 3.00 | 3.00 |
| Polyglyceryl-4 isostearate & Hexyl | | | |
| laurate | | | |
| Cetyl PEG/PPG-10/1 dimethicone | 2.00 | 2.00 | 2.00 |
| Magnesium sulphate | 0.60 | 0.60 | 0.60 |
| Phenoxyethanol & Caprylyl glycol & | 0.55 | 0.55 | 0.55 |
| Ethylhexylglycerin | | | |
| Aqua & Myrtus communis Leaf Extract | 1.00 | 2.00 | 2.50 |
| Aqua & Glycerin & Quercus Petraea | 2.00 | 3.00 | 1.00 |
| Fruit Extract | | | |
| Pinus Pinaster Bark Extract | 0.10 | 0.20 | 0.30 |

### Method of Manufacture

1. In the main vessel add Dimethicone, Dimethicone crosspolymer, PEG/PPG-18/18 dimethicone & polyglyceryl-4 isostearate & hexyl laurate and Cetyl PEG/PPG-10/1 dimethicone to make the oil phase.
2. Pre dissolve Pinus Pinaster Bark Extract in a small amount of water. Warm to 40-50° C. to dissolve.
3. Separately weigh out water, magnesium sulphate, glycerin, phenoxyethanol & caprylyl glycol & ethylhexylglycerin, Aqua & Myrtus communis Leaf Extract and Aqua & Glycerin & Quercus Petraea Fruit Extract stir until solids are dissolved to make the water phase. Add the dissolved Pinus Pinaster bark extract.
4. Add the water phase to the oil phase slowly with constant stirring at high speed (creating a vortex). Continue stirring for 5 minutes.
5. Homogenise the product for 5 minutes at 3500 rpm using a Silverson mixer or equivalent.

Example 2 - Oil in water emulsion

| Material | % w/w | % w/w | % w/w |
|---|---|---|---|
| Dimethicone | 5.50 | 5.50 | 5.50 |
| Water | 78.65 | 77.75 | 77.15 |
| Glycerin | 5.00 | 5.00 | 5.00 |
| Dimethicone crosspolymer & Dimethicone | 1.00 | 1.00 | 1.00 |
| Phenoxyethanol & Caprylyl glycol & | 0.80 | 0.80 | 0.80 |
| Ethylhexylglycerin | | | |
| Glyceryl stearate & PEG-100 stearate | 2.00 | 2.00 | 2.00 |
| Cetearyl alcohol | 2.00 | 2.00 | 2.00 |
| Sodium polyacrylate | 0.60 | 0.60 | 0.60 |
| Xanthan gum | 0.10 | 0.10 | 0.10 |
| Tetrasodium EDTA | 0.05 | 0.05 | 0.05 |
| Aqua & Myrtus communis Leaf Extract | 3.00 | 2.00 | 2.50 |
| Aqua & Glycerin & Quercus Petraea Fruit | 1.00 | 3.00 | 2.50 |
| Extract | | | |
| Pinus Pinaster Bark Extract | 0.30 | 0.20 | 0.80 |

### Method of Manufacture

1. To water add glycerin and dissolve tetrasodium EDTA.
2. Using homogenisation sprinkle in xanthan gum and continue to homogenise for 5 minutes or until hydrated.
3. Heat water phase to 70-75° C.
4. In a separate vessel weigh out oil phase and heat to 70-75° C. (Dimethicone, cetearyl alcohol, glyceryl stearate & PEG-100 stearate) When at temperature stir in the sodium polyacrylate.
5. With both phases at 70-75° C. add the oil phase to the water phase and homogenise for 2 minutes.
6. Add dimethicone crosspolymer & dimethicone and homogenise for 2 minutes.
7. Cool to room temperature.
8. Stir in Phenoxyethanol & Caprylyl glycol & Ethylhexylglycerin and Aqua & Myrtus communis Leaf Extract and Aqua & Glycerin & Quercus Petraea Fruit Extract.
9. Dissolve Pinus Pinaster Bark Extract in a small amount of water by warming to 40-50° C. and stir into the bulk.
10. Make to weight with water and stir smooth.

Example 3 - Oil in water emulsion containing sunscreens

| Material | % w/w | % w/w | % w/w |
|---|---|---|---|
| C12-15 alkyl benzoate | 5.00 | 5.00 | 5.00 |
| Butyl methoxydibenzoylmethane | 3.00 | 3.00 | 3.00 |
| Ethylhexyl methoxycinnamate | 5.00 | 5.00 | 5.00 |
| Cetearyl alcohol | 2.00 | 2.00 | 2.00 |
| Glycerin | 2.00 | 2.00 | 2.00 |
| Glyceryl stearate & PEG-100 stearate | 3.00 | 3.00 | 3.00 |
| Dimethicone | 1.50 | 1.50 | 1.50 |
| PEG-20 stearate | 0.50 | 0.50 | 0.50 |
| Phenoxyethanol & Caprylyl glycol & | 0.40 | 0.40 | 0.40 |
| Ethylhexylglycerin | | | |
| Carbomer | 0.20 | 0.20 | 0.20 |
| Potassium hydroxide | 0.06 | 0.06 | 0.06 |
| Tetrasodium EDTA | 0.05 | 0.05 | 0.05 |
| Water | 74.69 | 72.09 | 72.19 |
| Aqua & Myrtus communis Leaf Extract | 1.00 | 2.00 | 3.50 |
| Aqua & Glycerin & Quercus Petraea | 1.50 | 3.00 | 1.00 |
| Fruit Extract | | | |
| Pinus Pinaster Bark Extract | 0.10 | 0.20 | 0.60 |

### Method of Manufacture

1. To water add glycerine and dissolve tetrasodium EDTA.
2. Using homogenisation sprinkle in carbomer and continue to homogenise for 5 minutes or until hydrated.
3. Heat water phase to 70-75° C.
4. In a separate vessel weigh out oil phase and heat to 70-75° C. (Dimethicone, cetearyl alcohol, glyceryl stearate & PEG-100 stearate, C12-15 alkyl benzoate, Butyl methoxydibenzoylmethane, Ethylhexyl methoxycinnamate, PEG-20 stearate)
5. With both phases at 70-75° C. add the oil phase to the water phase and homogenise for 2 minutes.
6. Add Potassium hydroxide and homogenise for 2 minutes.
7. Cool to room temperature.
8. Stir in Phenoxyethanol & Caprylyl glycol & Ethyhexyglycerin and Aqua & Myrtus communis LeafExtract and Aqua & Glycerin & Quercus Petraea Fruit Extract.
9. Dissolve Pinus Pinaster Bark Extract in a small amount of water by warming to 40-50° C. and stir into the bulk.
10. Make to weight with water and stir smooth.

Example 4 - Gel

| Material | % w/w | % w/w | % w/w |
|---|---|---|---|
| Glycerin | 5.00 | 5.00 | 5.00 |
| Propanediol | 2.00 | 2.00 | 2.00 |
| Acrylates/C10-30 alkyl acrylate | 1.00 | 1.00 | 1.00 |
| crosspolymer | | | |
| Phenoxyethanol & Caprylyl glycol & | 0.40 | 0.40 | 0.40 |
| Ethylhexylglycerin | | | |
| Potassium hydroxide | 0.29 | 0.29 | 0.29 |
| Tetrasodium EDTA | 0.05 | 0.05 | 0.05 |
| Water | 86.96 | 86.06 | 86.16 |
| Aqua & Myrtus communis Leaf Extract | 0.50 | 2.00 | 2.70 |
| Aqua & Glycerin & Quercus Petraea | 3.50 | 3.00 | 1.50 |
| Fruit Extract | | | |
| Pinus Pinaster Bark Extract | 0.30 | 0.20 | 0.90 |

### Method of Manufacture

1. To water add glycerine and propanediol and dissolve tetrasodium EDTA.
2. Using homogenisation sprinkle in acrylates/C10-30 alkyl acrylate crosspolymer and continue to homogenise for 5 minutes or until hydrated.
3. Stir in potassium hydroxide to form gel.
4. Stir in Phenoxyethanol & Caprylyl glycol & Ethylhexylglycerin and Aqua & Myrtus communis Leaf Extract and Aqua & Glycerin & Quercus Petraea Fruit Extract.
5. Dissolve Pinus Pinaster Bark Extract in a small amount of water by warming to 40-50° C. and stir into the bulk.
6. Make to weight with water and stir smooth.

Example 5 - Detergent wash

| Material | % w/w | % w/w | % w/w |
|---|---|---|---|
| Sodium laureth sulphate | 36 | 36 | 36 |
| Cocamidopropyl betaine | 4 | 4 | 4 |
| Cocamide DEA | 1.5 | 1.5 | 1.5 |
| Sodium chloride | 1.5 | 1.5 | 1.5 |
| Sodium benzoate | 0.3 | 0.3 | 0.3 |
| Tetrasodium EDTA | 0.05 | 0.05 | 0.05 |
| Citric acid | 0.05 | 0.05 | 0.05 |
| Sodium hydroxide | 0.05 | 0.05 | 0.05 |
| Water | 53.00 | 51.35 | 49.65 |
| Aqua & Myrtus communis Leaf Extract | 1.35 | 2.00 | 3.00 |
| Aqua & Glycerin & Quercus Petraea | 2.00 | 3.00 | 3.00 |
| Fruit Extract | | | |
| Pinus Pinaster Bark Extract | 0.20 | 0.20 | 0.90 |

### Method of Manufacture

1. To water tetrasodium EDTA and sodium chloride.
2. Stir in sodium laureth sulfate, cocamidopropyl betaine, cocamide DEA.
3. Dissolve sodium benzoate in a small amount of water and stir into bulk,
4. Stir in Aqua & Myrtus communis Leaf Extract and Aqua & Glycerin & Quercus Petraea Fruit Extract.
5. Dissolve Pinus Pinaster Bark Extract in a small amount of water by warming to 40-50° C. and stir into the bulk.
6. Add sodium hydroxide and citric acid to achieve desired pH.
7. Make to weight with water and stir smooth

Additional details are provided below for some commercially available materials that are available and sold under the identified trade marks.

Table of commercially available materials

| | | | % | | |
|---|---|---|---|---|---|
| | | | active | | |
| Supplier | Trade name | Active INCI | INCI | Active compound | Excipients |
| Mibelle | Lipobelle ™ | Soy Isoflavones | | Genistein | Alcohol, lecithin, |
| | Soyaglycone | | | | Polsorbate 80 & |
| | | | | | Aqua. |
| DSM | Regu ®-Fade | Resveratrol | 100% | Trans resveratrol | None |
| Lipotec | Lipochroman ™ | Dimethylmethoxy | 100% | Dimethylmethoxy | None |
| | Molecule | chromanol | | chromanol | |

Table of commercially available Plant Extracts

| | | Active | % active | Active | | Extraction |
|---|---|---|---|---|---|---|
| Supplier | Trade name | INCI | INCI | compound | Excipients | solvent |
| Indena/ | Centevita ™ | Centella | 100% | Asiatic and | None | None |
| Givaudan | | asiatica leaf | | madecassic | | |
| | | extract | | acid 45 to | | |
| | | | | 100% | | |
| Croda | Phytessence ™ | Quercus | 5-10% | Polyphenols | Water 50-70%, | Ethanol/water |
| | French Oak | Petraea | | 0.70-1.10% | Glycerin 25- | |
| | | fruit extract | | | 50%, Sodium | |
| | | | | | benzoate | |
| | | | | | approx 0.6%, | |
| | | | | | Citric acid | |
| | | | | | approx 0.04% | |
| Merck | Emblica ® | Phyllanthus | 100% | Contains min. | 100% pure | |
| | | Emblica | | 60% low | | |
| | | fruit Extract | | molecular | | |
| | | | | weight | | |
| | | | | hydrolysable | | |
| | | | | tannins, | | |
| | | | | Emblicanin A | | |
| | | | | & Emblicanin | | |
| | | | | B being the | | |
| | | | | key bio-active | | |
| | | | | ingredients | | |
| Clariant | Red Snow ® | Camellia | 1-5% | Protocatechuic | Propanediol 30- | Propanediol/ |
| | | Japonica | | acid min. | 70%, Aqua 30- | water |
| | | flower | | 200 ppm | 70% | |
| | | extract | | | | |
| Naturex | Botany | Ginkgo | 100% | Ginkgoflavon- | 100% pure | Hydroalcoholic |
| | Ginkgo ™ | Biloba leaf | | glucosides 24- | | |
| | | extract | | 30%, Terpene | | |
| | | | | lactones 6-9% | | |
| Silab | Desoxine ® | Myrtus | 2.65% | polyphenols | water 97.05%, | |
| | Bio | Communis | | 2.5 g/l-5.0 g/l | Sodium | |
| | | leaf extract | | | benzoate 0.3% | |
| BASF | Arganyl ® LS | Argania | Approx. | Flavonoides | Glycerin | Glycerin/ |
| | 9781 | Spinosa leaf | 5.00% | 0.30-0.45% | 47.00%, | water |
| | | extract | | | Phenoxyethanol | |
| | | | | | 2.00%, Water | |
| | | | | | to 100% | |
| Horphag | Pycnogenol ® | Pinus | 100% | Procyanidins | 100% pure | |
| Research | | Pinaster | | 65-75% | | |
| | | back extract | | | | |
| Naturex | Green Tea | Camellia | 100% | Polyphenols >95%/ | 100% pure | n/a |
| | PE ™ | sinensis | | Catechins >70% | | |
| | | leaf extract | | | | |
| Silab | Cohesium ® | Ophiopogon | 9-13% | Sugars | Water | Water |
| | | japonicus | | (Fructose): 85- | | |
| | | root extract | | 127 g/L | | |

1. A cosmetic composition comprising the antioxidant agents: Myrtus communis leaf extract, Quercus petraea fruit extract and Pinus pinaster bark extract.

2. The cosmetic composition of claim 1 wherein the cosmetic composition further comprises one or more additional antioxidant agents.

3. The cosmetic composition of claim 1, wherein the Myrtus communis leaf extract is present in an amount of about 0.0001% to about 1% by weight of the composition.

4. The cosmetic composition of claim 1, wherein the Myrtus communis leaf extract is present in an amount of about 0.001% by weight of the composition.

5. The cosmetic composition of claim 1, wherein the Quercus petraea fruit extract is present in an amount of about 0.0001% to about 1% by weight of the composition.

6. The cosmetic composition of claim 1, wherein the Quercus petraea fruit extract is present in an amount of about 0.001% by weight of the composition.

7. The cosmetic composition of claim 1, wherein the Pinus pinaster bark extract is present in an amount of about 0.0001% to about 1% by weight of the composition.

8. The cosmetic composition of claim 1, wherein the Pinus pinaster bark extract is present in an amount of about 0.001% by weight of the composition.

9. The cosmetic composition of claim 1 wherein at least two of the antioxidant agents are present in equal amounts.

10. The cosmetic composition of claim 1 wherein equal amounts of Myrtus communis leaf extract, Quercus petraea fruit extract and Pinus pinaster bark extract are present.

11. The cosmetic composition of claim 1 wherein the cosmetic composition is for topical application.

12. A method of cosmetic treatment of a skin/hair condition comprising the step of applying a cosmetic composition as defined in claim 1 onto the skin/hair of a subject in need thereof.

13. (canceled)
